(19) United States
(12) Patent Application Publication
(10) Pub. No.: US 2021/0221862 A1
(43) Pub. Date: Jul. 22, 2021

(54) APPLICATION OF PEDF-DERIVED SHORT PEPTIDES IN THE TREATMENT OF OSTEOARTHRITIS

(71) Applicant: BRIM Biotechnology, Inc., Taipei (TW)

(72) Inventors: Frank Wen-Chi Lee, Bedford, MA (US); Yuan-Ming Lee, Taipei (TW); Yeou-Ping Tsao, Taipei (TW); Tsung-Chuan Ho, Taipei (TW)

(73) Assignee: BRIM Biotechnology, Inc., Taipei (TW)

(21) Appl. No.: 17/053,058

(22) PCT Filed: Apr. 8, 2019
(86) PCT No.: PCT/US2019/026347

§ 371 Date: Nov. 4, 2020

Related U.S. Application Data
(60) Provisional application No. 62/654,468, filed on Apr. 8, 2018.

(51) Int. Cl. C07K 14/475; A61P 19/02; C07K 7/08

(52) U.S. Cl. CPC C07K 14/475; A61K 38/00; C07K 7/08; A61P 19/02

(57) ABSTRACT

A method for treating and/or preventing osteoarthritis includes administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

### FIELD OF THE INVENTION

This invention relates to PEDF-derived peptides and their uses in tendon healing after injuries.

### BACKGROUND OF THE INVENTION

Osteoarthritis (OA), the most common type of joint disease, is a degenerative disorder resulting from breakdown of articular cartilage in synovial joints. With age, articular cartilage degenerates at the cellular level (i.e., chondrocyte). There is a decrease in the numbers of chondrocytes and proteoglycans, leading to an overall loss of cartilage thickness. A breakdown in the structure and function of articular chondrocytes (AC) leads to osteoarthritis that affects millions of people worldwide. However, AC has a limited ability to self-heal in larger trauma due to the avascular nature and the resting nature of articular chondrocytes (Khan et al., 2008, “Cartilage integration: evaluation of the reasons for failure of integration during cartilage repair,” Eur. Cell. Mater., 2008 Sep. 3; 16:26-39). In addition, cartilage is frequently injured, such as in sports-related trauma. Therefore, chondral defects and early osteoarthritis represent a major challenge for orthopedic surgeons.

Osteoarthritis is a progressive, heterogeneous, degenerative joint disease, and the most common form of arthritis, especially in older people. Osteoarthritis is associated with a breakdown of cartilage in joints and can occur in almost any joint in the body. It commonly occurs in the weight bearing joints of the hips, knees, and spine, but can also affect fingers, neck, and large toes. However, osteoarthritis rarely affects other joints unless prior injury or excessive stress is involved. Loss of articular cartilage through injury or disease presents major clinical challenges.

Chondrocytes in cartilage differentiates from mesenchymal cells during embryonic development. Differentiated chondrocytes, which are the only cell type found in a normal mature cartilage, synthesize sufficient amounts of cartilage-specific extracellular matrix (ECM) to maintain matrix integrity. The primary constituents of ECM are water, aggrecans, and type II collagen that resists applied compressive forces generated by locomotion of the underlying bone.

Treatment options for OA are very limited. They include analgesics, non-steroidal anti-inflammatory drugs (NSAIDs), and intra-articular injections of steroids or hyaluronan (HA; to improve joint lubrication). Physical therapy is an option. Surgical options range from arthroscopic procedures to total joint arthroplasty. In addition, allograft transplant by surgical procedure is being developed. These limited treatment options may provide some relief. However, there is still a need for better treatments for osteoarthritis.

### SUMMARY OF INVENTION

Embodiments of the invention relate to methods for treating and/or preventing osteoarthritis using pigmented epithelia derived factor (PEDF)-derived short peptides. Some embodiments of the invention relate to methods for promoting chondrogenesis.

One aspect of the invention relates to pharmaceutical composition or methods for treating and/or preventing osteoarthritis. A method in accordance with embodiments of the invention includes administering to a subject in need thereof a pharmaceutical composition comprising a PEDF-derived short peptide (PDSP) or a variant of the PDSP, wherein the PDSP comprises residues 93-106 of the human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF. The PDSP comprises the sequence of the sequences of any one of SEQ ID NO: 1 to 75.

One aspect of the invention relates to pharmaceutical composition or methods for promoting chondrogenesis. A method in accordance with embodiments of the invention comprises contacting multipotent mesenchymal stem cells with a composition comprising a PEDF-derived short peptide (PDSP) or a variant of the PDSP, wherein the PDSP comprises residues 93-106 of the human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF. The PDSP comprises the sequence of the sequences of any one of SEQ ID NO: 1 to 75.

Other aspect of the invention will become apparent with the following detailed description and the attached claims.

### BRIEF DESCRIPTION OF THE DRAWINGS

FIG. 1 shows the effects of 29-mer and hyaluronic acid on rat model of MIA-induced changes in the hind paw weight-bearing distributions. Rats were injected with 1 mg of monoiodoacetate (MIA) in the right (osteoarthritic) knees and saline in the left (contralateral control) knees. The 29-mer and 1% HA treatments were conducted at day 8 post-MIA injection for further 2 weeks. Changes in hind paw weight distributions (weight bearing) were assessed by use of an incapacitance tester. Values given represent the average±SE from at least 3 rats from each treatment group. *P<0.05 versus untreated group.

FIG. 2 shows results of histological analysis of the 29-mer PDSP (PEDF-derived short peptide) effects on MIA-damaged articular cartilage. Rat knee joints were injected once with MIA. The vehicle/HA and 29-mer/HA treatments were conducted at day 8 post-MIA injection for further 2 weeks. Representative photomicrographs of H&E-stained sections of articulate cartilage from three independent experiments. F: femoral condyle; T: tibial condyle, M: meniscus. * indicate the femorotibial joints and the magnification view of lateral tibial cartilage in right panel. Arrows indicate the necrotic chondrocytes.

FIG. 3 shows results of semiquantitative analysis of glycosaminoglycan (GAG)-rich extracellular matrix by Alcian blue staining after chondrogenic differentiation of rat MSCs for 3 weeks. MSCs in chondrogenic differentiation medium supplemented with different 29-mer variants (10 μM) for 14 days. OD values of Alcian blue extracted by guanidinium chloride are shown relative to the total DNA contents of micromass. Data are represented as the mean±SE. Black and gray columns, representing OD values 0.16 and 0.25, respectively, indicate mutations that completely and partially impaired chondrogenic-promoting activities of the 29-mer.

FIG. 4 shows effects of 29-mer variants on rat model of MIA-induced changes in the hind paw weight-bearing distribution. Rats injected with 1 mg of MIA in the right (osteoarthritic) knees and saline in the left (contralateral control) knees. The 29-mer/HA, 29-mer variant/HA, and vehicle/HA treatments were conducted at day 8 post-MIA injection for further 2 weeks. Changes in hind paw weight bearings were assessed by use of an incapacitance tester. Values given represent the average±SE from at least 3 rats from each treatment group.

FIG. 5 shows results of PDSP-induced chondrogenic cell proliferation in damaged articular cartilage in a dose-dependent manner. (A) Upper panels: histological analysis of cell replication at day 14 after the 29-mer treatment. Specimens were stained with BrdU to indicate DNA replication (deep brown color). Original magnification, 200×. Lower panels: Representative pictures showing expressions of Sox9 (green; a marker of chondrocytes) and BrdU (red) in articular cartilages assayed by dual-immunostaining. Original magnification, 1000×. (B) Numbers of BrdU-positive cells per field of cartilage region evaluated. *P<0.001 versus vehicle/HA group.

FIG. 6 shows mitogenic effects of 29-mer variants on damaged articular cartilage in rat model of MIA-induced OA. Rats injected with 1 mg of MIA in the right (osteoarthritic) knees and saline in the left (contralateral control) knees. The 29-mer/HA, 29-mer variant/HA, and vehicle/HA treatments were conducted at day 8 post-MIA injection for further 2 weeks. Knee joints were stained with BrdU to identify proliferating cells. BrdU-positive cells per field of view on cartilage region of knee joint sections were counted (Original magnification×100). Total BrdU⁺ cells were evaluated from 6 sections/knee joint specimen, with 3 rats in each group.

### DETAILED DESCRIPTION

Embodiments of the invention relates methods for preventing and/or treating osteoarthritis using PEDF-derived short peptides (PDSP). The invention is based on unexpected findings that certain short peptides derived from pigmented epithelia-derived factor (PEDF) can alleviate pains in osteoarthritis and confer articular cartilage repairs by inducing mesenchymal cell differentiation to form chondrocytes.

Osteoarthritis is a degenerative disorder resulting from breakdown of articular cartilage (AC) in synovial joints. However, AC has a limited ability to self-heal due to the avascular nature and the resting nature of articular chondrocytes. Normal mature cartilage comprises chondrocytes, which differentiate from mesenchymal cells during embryonic development. Differentiated chondrocytes, which are the only cell type found in normal mature cartilage, synthesize sufficient amounts of cartilage-specific extracellular matrix (ECM) to maintain matrix integrity.

Human Pigment Epithelium-derived Factor (PEDF) is a secreted protein containing 418 amino acids, with a molecular weight of about 50 kDa. PEDF is a multifunctional protein with many biological functions (see e.g., U.S. Patent Application Publication No. 2010/0047212). Different peptide regions of the PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotrophic properties.

Inventors of the present invention found that certain short peptides of PEDF can alleviate pains in osteoarthritis. It was further found that the pain reduction arises from the abilities of these PDSPs to induce cartilage regeneration. The inventors showed that the PDSPs can induce multipotent mesenchymal stem cells (MSC) that are present in or around the cartilage to differentiate into chondrocytes. That is, these PDSPs can promote chondrogenesis. This may explain the abilities of the PDSPs to induce cartilage regeneration and pain reduction.

As noted above, differentiation of mesenchymal cells into chondrocytes normally occurs in embryonic development. In cartilage, mesenchymal stem cells lose their pluripotency and proliferate to form a dense aggregate of chondrogenic cells, which then differentiate into chondroblasts, which synthesize the cartilage extracellular matrix (ECM). The chondroblasts become mature chondrocytes that are usually inactive but can still secrete and degrade the matrix, depending on conditions. Therefore, the finding that PDSP can induce mesenchymal cells (in or around cartilage) to produce chondrocytes in cartilage is truly unexpected.

The PDSPs of the invention are based on the peptide region corresponding to human PEDF residues 93-121 (⁹³SLGAEQRTESIIHRALYYDLISSPDIHGT¹²¹; SEQ ID NO:1). Based on this 29-mer, inventors identified that serine-93, alanine-96, glutamine-98, isoleucine-103, isoleucine-104, and arginine-106 are critical for the activities, as evidenced by significant loss of activities when these residues were individually replaced with alanine (or glycine for Alanine-96). In contrast, alanine (or glycine) replacements of other residues in the 29-mer did not appreciably change the activities, suggesting PDSP variants having amino acid substitutions (particularly, homologous amino acid substitutions) at these other residues (i.e., residues 94, 95, 97, 99-102, 105, and 107-121) can also be used to prevent and/or treat osteoarthritis, or to induce chondrogenesis.

These results indicate that the core peptide containing the antinociceptive effects is in the region comprising residues 93-106 (⁹³SLGAEQRTESIIHR¹⁰⁶; SEQ ID NO:2). Thus, the shortest PDSP peptide having the antinociceptive activity may be a 14-mer. One skilled in the art would appreciate that addition of additional amino acids to this core peptide, at the C and/or N terminus, should not affect this activity. That is, a PDSP of the invention may be any peptide comprising residues 93-106 of human PEDF. Therefore, a PDSP peptide for the invention may be a 14-mer, 15-mer, 16-mer, and so on, including the 29-mer used in the experiments.

Furthermore, as noted above, substitutions within these short peptides can retain the activities, as long as the critical residues (serine-93, alanine-96, glutamine-98, isoleucine-103, isoleucine-104, and arginine-106) are preserved. In addition, the mouse variants (which have two substitutions: histidine-98 and valine-103, as compared with the human sequence) are also active. The corresponding mouse sequences are: mo-29mer (SLGAEHRTESVIHRALYYDLITNPDIHST, SEQ ID NO: 3) and mo-14mer (SLGAEHRTESVIHR, SEQID NO: 4). Thus, a generic sequence for an active core is (⁹³S-X-X-A-X-Q/H-X-X-X-X-I/V-I-X-R¹⁰⁶, wherein X represents any amino-acid residue; SEQ ID NO: 5).

PDSP peptides of the invention may be chemically synthesized or expressed using protein/peptide expression systems. These PDSP peptides may be used in a pharmaceutical composition for the prevention and/or treatment of osteoarthritis. The pharmaceutical composition may comprise any pharmaceutically acceptable excipient, and the pharmaceutical composition may be formulated in a form suitable for administration, such as topical application, oral application, injection, etc. Various formulations for such applications are known in the art and can be used with embodiments of the invention.

Some embodiments of the invention relate to methods for treating and/or preventing osteoarthritis in a subject (e.g., human, pets, or other subjects). As used herein, the term “treat” or “treating” includes partial or total improvement of the condition, which may or may not include total cure. The method may comprise administering a pharmaceutical composition to the subject, wherein the pharmaceutical composition comprises an effective amount of a PDSP of the invention (including active variants of the PDSP). One skilled in the art would appreciate that the effective amount would depend on the conditions of the subject (e.g., weight, age, etc.), the route of administration, and other factors. Finding such effective amount involves only routine techniques and one skilled in the art would not require inventive efforts or undue experimentation to find the effective amount.

Embodiments of the invention will be illustrated with the following specific examples. In specific examples, the 29mer (SEQ ID NO:1) are used. However, other PDSP (e.g., 14mer, SEQ ID NO:2 or SEQ ID NO:3, etc.) can also be used to achieve the same results. One skilled in the art would appreciate that these examples are for illustration only and that variations and modifications are possible without departing from the scope of the invention.

### Materials and Methods

Dulbecco's modified Eagle's medium (DMEM), fetal bovine serum (FBS), 0.25% trypsin, and antibiotics were purchased from Invitrogen (Carlsbad, Calif., USA). Hyaluronic acid (HA), mono-iodoacetate (MIA), dimethyl sulfoxide (DMSO), Percoll, insulin, hydrocortisone, bovine serum albumin (BSA), 5-bromo-2′-deoxyuridine (BrdU), Hoechst 33258 dye, and Alcian blue 8-GX were all from Sigma-Aldrich (St. Louis, Mo., USA). Anti-BrdU and anti-SOX9 antibodies were from GeneTex (Taipei, Taiwan). All the fluorescent dye-conjugated secondary antibodies were purchased from BioLegend (San Diego, Calif., USA). Hematoxylin and eosin (H&E) dyes were purchased from Merck (Rayway, N.J., USA). Synthetic PEDF peptides were synthesized and modified with acetylation at the NH₂ termini and/or amidation at the COOH termini for stability. The synthetic PEDF peptides were characterized with mass spectrometry (>95% purity) at GenScript (Piscataway, N.J.). Each PEDF-derived synthetic peptide was reconstituted in DMSO as stock (5 mM).

All animals used in the present studies were housed in an animal room under temperature control (24-25° C.) and a 12:12 light-dark cycle. Standard laboratory chow and tap water were available ad libitum. The experimental procedures were approved by the Mackay Memorial Hospital Review Board (New Taipei City, Taiwan, R.O.C.) and were performed in compliance with Taiwan national animal welfare regulations.

### Animal Osteoarthritis Model and Treatments

Adult 10-wk-old male Sprague-Dawley rats (initial body wt=312±11 g) were anesthetized by an intraperitoneal injection of Xylazine (10 mg/kg). After that, their right knees were each treated with a single intraarticular injection of 1 mg of MIA in 25 μl of sterile saline. The solution was injected through the patellar ligament using a 27 G needle with the leg flexed at a 90° angle at the knee. Seven days after MIA injection, the mice were randomly assigned to different experimental groups (n 3, each group). For treatments of the rat model of MIA-induced OA, PDSP peptide was dissolved in 25 μl of 1% HA, and the peptide solvent DMSO was used as a vehicle/HA control.

### Assessment of changes in hind paws weight distributions

Changes in hind paw weight distributions between the right (osteoarthritic) and left (contralateral control) limbs were used as an index of joint discomfort in the osteoarthritic knee. An incapacitance tester was employed for determination of hind paw weight distributions, as previously described (Bove et al., Weight bearing as a measure of disease progression and efficacy of anti-inflammatory compounds in a model of monosodium iodoacetate-induced osteoarthritis. Osteoarth Cart., 2003; 11:821-830). Rats were placed in an angled plexiglass chamber positioned so that each hind paw rested on a separate force plate. The force exerted by each hind limb (measured in grams) is averaged over a 5-s period. Each data point is the mean of three, 5-s readings. The change in hind paw weight distribution was calculated by determining the difference in the amounts of weights (g) exerted on the tester between the left and right limbs. Results are presented as either the difference in weight bearing between the left (contralateral control) limb and right (osteoarthritic) limbs or as the percent difference between the baseline reading and the post-treatment reading, as calculated using the following equation:

(1−(mean Δweight of treated group/mean Δ weight of vehicle group))×(100)

### Isolation and Culture of Mesenchymal Stem Cells (MSCs)

Adult 8-wk-old male Sprague-Dawley rats were anesthetized with an intraperitoneal injection of Xylazine (10 mg/kg). After that, their femurs were aseptically harvested, washed in a mixture of PBS and antibiotics for 5 minutes, and then they were dissected off of all soft tissue, transected at their epiphysis, and their marrow cavity rinsed repeatedly with a mixture of heparin (AGGLUTEX INJ 5000 U/ML 5 ML; working conc. 100 U/ml) and DMEM. The harvested cells were collected, dispersed by pipetting, and centrifuged at 1000×g for 5 minutes at RT. Cell pellets were resuspended with DMEM, and then the cell suspension was transferred to a 15-ml centrifuge tube containing 5 ml of Percoll (1.073 g/ml). After centrifugation at 1500×g for 30 minutes, the mononuclear cells in the middle layer were obtained, washed three times with PBS, and then suspended in low-glucose DMEM with 10% heat-inactivated FBS and 1% penicillin/streptomycin. Cells were then placed in 75-cm² flasks (Corning, Mass., USA) and incubated with 95% air and 5% CO₂ at 37° C. The medium was replaced every 4 d. Unattached cells were discarded and adherent cells were retained. The primary MSCs grew to approximately 80%-90% confluence after culturing for 1 week.

### Chondrogenic Differentiation of MSCs

5×10³ expanded MSCs were placed in each well of a 96-well plate and exposed to 150 μl of chondrogenic medium (high-glucose DMEM with 100 nM dexamethasone, 0.17 mM ascorbic acid-2 phosphate, 10 μg/ml of insulin, 5 μg/ml of transferrin, 5 ng/ml selenium, 1 mM sodium pyruvate, 2 mM L-glutamine, and 2% FBS) supplemented with 10 ng/ml TGF-β3 (R&D Systems, Minneapolis, Minn., USA), and 10 μM PDSP peptide. The medium was replaced every 3 days, and the cells were cultivated for 2 weeks.

### Histological Examination of the Knee Joint

The knee joints were dissected, and the surrounding soft tissue was removed. Specimens were fixed in a 4% paraformaldehyde (PFA) solution and then were decalcified with Shandon TBD-2 decalcifier (Thermo Scientific, Logan, Utah). The joints were then sectioned mid-sagittally and embedded in paraffin blocks. Sections (5 μm in thickness) were longitudinally cut and stained with hematoxylin and eosin (H&E) or used for immunohistochemical examination. 20 sections per knee were carefully prepared so as to include the most severely degenerated area.

### In Vivo Detection of DNA Synthesis

For detection of cell proliferation, BrdU was reconstituted in DMSO as stock (80 mM). 150 μl of BrdU mixed with 350 μl of PBS was intraperitoneally injected into rat at day 1, 4, and 8 after MIA injection for 7 days (i.e., day 7 after MIA inject set as day 0). DNA synthesis was assessed by BrdU labeling, as detected with anti-BrdU antibodies.

### Immunofluorescence and BrdU Staining

To detect DNA synthesis in vivo, paraffin-embedded joint specimens were deparaffinized in xylene and rehydrated in a graded series of ethanol and then exposed to 1 N HCl at RT for 1 h for subsequent immunohistochemistry. The tissue sections were then blocked with 10% goat serum and 5% BSA for 1 h. Immunostaining was done using primary antibodies against SOX9 (1:100 dilution) and BrdU (1:100 dilution) at 37° C. for 2 h, followed by incubation with an appropriate rhodamine- or FITC-conjugated donkey IgG for 1 h at RT. Nuclei were located by counterstaining with Hoechst 33258 for 7 min. Images were captured using a Zeiss epifluorescence microscope with a CCD camera and measured from 20 randomly-selected areas in each sample, and blind quantification was performed in triplicate by manually counting within each section.

Deparaffinized knee joint specimens were also blocked with 10% goat serum for 60 min and then incubated with an antibody against BrdU. The slides were subsequently incubated with an appropriate peroxidase-labeled goat immunoglobulin (1:500 dilution; Chemicon, Temecula, Calif.) for 20 min and then incubated with chromogen substrate (3,3′-diaminobenzidine) for 2 min before counterstaining with hematoxylin.

### Alcian Blue Staining and Quantification

For Alcian blue staining, cultures were rinsed twice with PBS, fixed in 4% (w/v) paraformaldehyde for 15 min, and then incubated in 1% (w/v) Alcian blue 8-GX (Sigma) in 0.1 N HCl (pH 1.0) overnight as previously described (Ji Y. H. et al., Quantitative proteomics analysis of chondrogenic differentiation of C3H10T1/2 mesenchymal stem cells by iTRAQ labeling coupled with on-line two-dimensional LC/MS/MS, Mol. Cell. Proteom., 2010, 9(3): 550-564.). For semi-quantitative analysis, Alcian blue-stained cultures were extracted with 6 M guanidine HCl for 2 h at room temperature. The absorption of the extracted dye was measured at 650 nm in a microplate reader (Bio-Rad). To measure DNA contents, 100 μL of the extracts were combined with 100 μL of 0.7 μg/ml Hoechst 33258 (Sigma-Aldrich) in water. Fluorescence was read with Ex/Em: 340 nm/465 nm and compared to that of a certified calf thymus sonicated DNA standard (Sigma-Aldrich).

### Statistics

Results were expressed as mean±standard error of the mean (SEM). 1-way ANOVA was used for statistical comparisons. P<0.05 was considered significant, unless otherwise specified.

### PDSP Exhibits Antinociceptive Effects to Ameliorate Joint Discomfort in OA Animal

Knee osteoarthritis (OA) is a common chronic degenerative disease characterized by loss of articular cartilage. Injection of MIA, an inhibitor of glycolysis, into the femorotibial joint space of rodents has been reported to induce loss of AC similar to that noted in human OA (Bove et al., 2003). In addition, it has been established that injection of MIA into the rat knee joint resulted in a dose and time-dependent increase in joint discomfort defined by the hind paw weight-bearing shift from the MIA-injected limb.

To investigate whether PDSP (PEDF short peptide) has antinociceptive effects to ameliorate the joint discomfort, 10-wk-old male Sprague-Dawley rats (n=15) were subjected to intraarticular injection of 1 mg of MIA (dissolved in 25 μl of sterile saline) into right joint to induce a maximum degree of weight shift (from right to left leg) and then used to investigate the pharmacologic response of the 29-mer PDSP (SEQ ID NO:1). After MIA injection for 7 days (set as day 0), the mice were randomly assigned to 4 experimental groups (n=3, each group) and treated for further 14 days as follows: (I) PDSP vehicle resolved in 25 μl of 1% hyaluronic acid (HA) mixed with PDSP vehicle, (II) PDSP 29-mer/HA (final concentration 0.2 mM PDSP with 1% HA), (III) PDSP 29-mer alone (bolus). Treatments were applied by way of a single intraarticular injection once on day 1, 4, 8, and 12 (twice per week). To test the therapeutic effects with decreased treatment frequency, group IV (29-mer/HA) was subjected to intraarticular injection once on day 1 and once on day 8 (i.e., once per week).

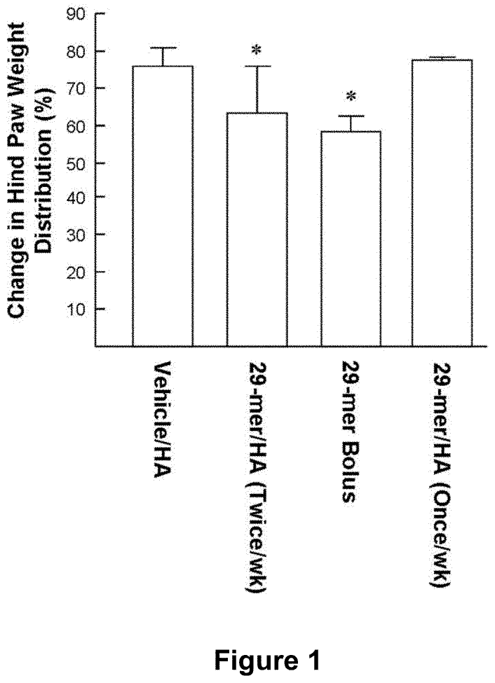

As shown in FIG. 1, the results revealed that 29-mer PDSP treatments significantly reduced the MIA-induced weight shifts, as compared to vehicle/HA group (group II and IV versus group I: 63.3±12.5% and 58.1±4.6% versus 75.9±4.7%; P<0.05). On the other hand, bolus injection groups were unable to decrease MIA-induced changes in the hind paw weight-bearing distribution (77.2±1.2%). These results indicate that the 29-mer PDSP combining with hyaluronic acid exhibits antinociceptive effects on MIA-induced joint discomfort in rats. The 29-mer PDSP bolus injection result also implies that the 29-mer PDSP may leak rapidly into the systemic circulation in the absence of hyaluronic acid.

It has been established that rat knee joints injected with MIA can result in extensive chondrocyte degeneration/necrosis at day 7 post-MIA treatment in a rapid and reproducible manner. Therefore, we further investigated the histopathologic features of femorotibial joints in rats from the vehicle/HA treatment group and 29-mer/HA treatment group.

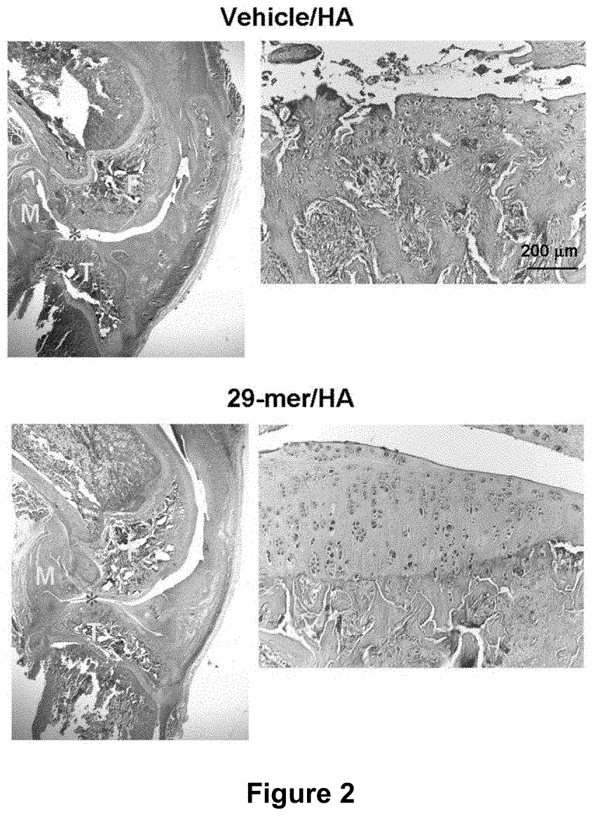

As depicted in FIG. 2, the vehicle/HA treatment group showed loss of cartilage integrity and subchondral bone collapse in the lateral tibia, whereas the 29-mer/HA treatment group revealed a good surface continuity. Microscopically, the vehicle/HA treatment group showed that chondrocytes were lost from the superficial zone of cartilage and scattered cell clusters occurred in transitional zone and radial zone extensively. In contrast, the 29-mer/HA treatment group showed that the occupation of large numbers of newly generated chondrocytes throughout the cartilage. The histological data suggest that the ability of the 29-mer PDSP to induce cartilage regeneration may be in part responsible for reducing OA pains.

### The 29-Mer Promotes Chondrogenic Activity of MSCs and Chondrogenic Cell Proliferation in Vivo

The chondrogenic potential of multipotent mesenchymal stem cells (MSCs) makes them a promising source for cell-based therapy of cartilage defects (M. F. Pittenger et al., 1999, Multilineage potential of adult human mesenchymal stem cells, Science, 284(5411): 143-7). Moreover, resident MSCs in response to cartilage injury might be induced to undergo chondrogenic differentiation for cartilage healing (T. B. Kurth et al., 2011, Functional mesenchymal stem cell niches in adult mouse knee joint synovium in vivo, Arthritis Rheum., 63(5): 1289-300). In culture, our data showed that pigment epithelium-derived factor (PEDF)-derived short peptide (29-mer; positions Ser93-Thr121) exhibited in vitro chondrogenic promoting activity on MSCs in the presence of a defined medium containing 100 nM dexamethason and 10 ng/ml TGF-03.

### Alanine Scanning Data of the 29-Mer PDSP with a Single Alanine or Glycine Alteration

In this study, single residue substitutions to alanine or glycine along the 29-mer sequence were designed and synthesized to dissect the critical residues in the 29-mer for the chondrogenic promoting activity. In total, 29 peptides variants were synthesized based on the amino acid sequence of PEDF positioned 93-121, including 27 variants with a single alanine alteration and 2 variants with a single glycine alteration (A96G and A107G). To evaluate the chondrogenesis of rat MSCs in culture treated with dexamethason, TGF-03, and 29-mer variants, the expressing levels of glycosaminoglycans (GAGs), a marker of mature chondrocytes, were detected by Alcian blue staining.

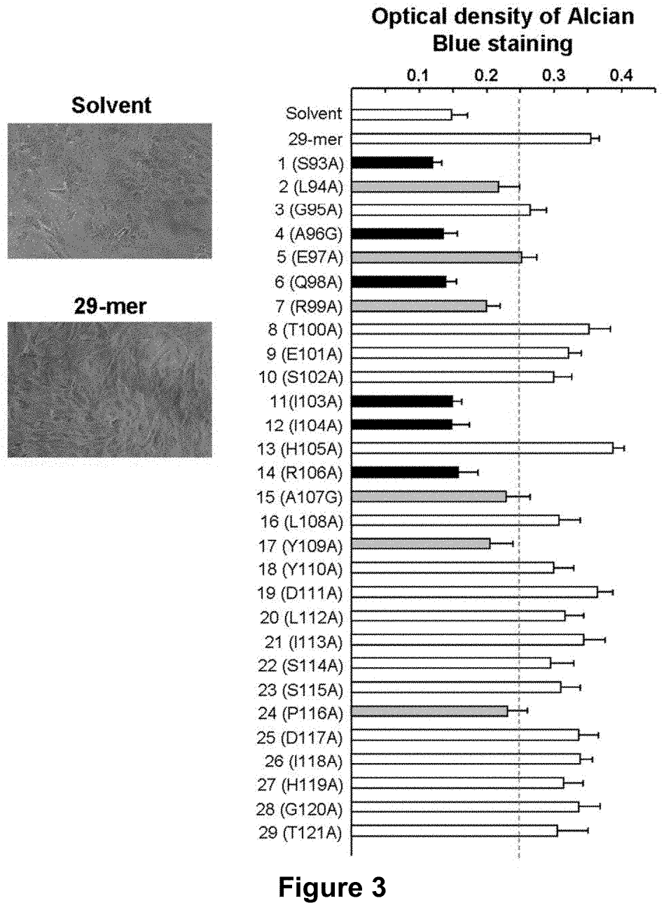

As shown in FIG. 3, MSCs in defined medium (containing dexamethason and TGF-β3) were exposed to 10 μM 29-mer variants for 21 days, followed with analysis of sulfated GAGS using Alcian blue staining. The results showed a clear increase in the staining intensity in culture of MSCs treated with 29-mer PDSP, as compared to DMSO solvent control, as evidenced by quantifying Alcian blue-positive material at OD 650 nm (0.34±0.013 versus 0.15±0.024). The results also revealed that S93A (0.12±0.015), A96G (0.14±0.023), Q98A (0.14±0.017), 1103A (0.15±0.013), 1104A (0.15±0.027%), and R106A (0.16±0.029) mutations severely impaired the chondrogenic promoting activity of the 29-mer PDSP on MSCs (0.12-0.16 versus 0.34). These results suggest that 6 out of 29 amino acids may critical for the 29-mer activity.

In addition, L94A (0.22±0.032), E97A (0.25±0.023), R99A (0.2±0.02), A107G (0.23±0.035), and P116A (0.23±0.029) mutations caused partially reduction in the chondrogenic promoting activity of 29-mer PDSP (O.D. 0.2-0.25 versus 0.34). The remaining substitutions did not substantially affect the chondrogenic promoting activity of the 29-mer PDSP (O.D.>0.26).

Collectively, alanine scanning data indicate that the chondrogenic promoting effect of the 29-mer (SEQ ID NO:1) on MSCs is influenced by the amino acid substitution and the core peptide is a 14mer (SEQ ID NO:2). Moreover, in terms of inducing MSC chondrogenic differentiation, the 29-mer PDSP at positions 93, 96, 98, 103, 104, and 106 cannot be substituted without affecting its function. On the other hand, the remaining amino acid residues in the 29-mer PDSP sequence displayed a greater flexibility with respect to single amino acid substitutions without affecting the 29-mer PDSP function. Thus, a minimal core peptide may be represented as ⁹³S-X-X-A-X-Q/H-X-X-X-X-I/V-I-X-R¹⁰⁶, wherein X represents any amino-acid residue (SEQ ID NO:5). A few examples of PDSP sequence that may be used with embodiments of the invention are shown in the following Table (the positions numberings are based on the positions in the 14mers). These examples are not meant to be limiting.

| | SEQ |
|---|---|
| | ID |
| Peptide Sequences | NO |
| ¹S-²X-³X-⁴A-⁵X-⁶Q/H-⁷X-⁸X-⁹X-¹⁰X-¹¹I/V-¹²I-¹³X-¹⁴R | 5 |
| ¹S-²L-³X-⁴A-⁵X-⁶Q/H-⁷X-⁸X-⁹X-¹⁰X-¹¹I/V-¹²I-¹³X-¹⁴R | 6 |
| ¹S-²A-³X-⁴A-⁵X-⁶Q/H-⁷X-⁸X-⁹X-¹⁰X-¹¹I/V-¹²I-¹³X-¹⁴R | 7 |
| ¹S-²X-³G-⁴A-⁵X-⁶Q/H-⁷X-⁸X-⁹X-¹⁰X-¹¹I/V-¹²I-¹³X-¹⁴R | 8 |
| ¹S-²X-³A-⁴A-⁵X-⁶Q/H-⁷X-⁸X-⁹X-¹⁰X-¹¹I/V-¹²I-¹³X-¹⁴R | 9 |
| ¹S-²X-³X-⁴A-⁵E-⁶Q/H-⁷X-⁸X-⁹X-¹⁰X-¹¹I/V-¹²I-¹³X-¹⁴R | 10 |
| ¹S-²X-³X-⁴A-⁵A-⁶Q/H-⁷X-⁸X-⁹X-¹⁰X-¹¹I/V-¹²I-¹³X-¹⁴R | 11 |
| ¹S-²X-³X-⁴A-⁵X-⁶Q/H-⁷R-⁸X-⁹X-¹⁰X-¹¹I/V-¹²I-¹³X-¹⁴R | 12 |
| ¹S-²L-³X-⁴A-⁵X-⁶Q/H-⁷A-⁸X-⁹X-¹⁰X-¹¹I/V-¹²I-¹³X-¹⁴R | 13 |
| ¹S-²A-³X-⁴A-⁵X-⁶Q/H-⁷X-⁸A-⁹X-¹⁰X-¹¹I/V-¹²I-¹³X-¹⁴R | 14 |
| ¹S-²X-³G-⁴A-⁵X-⁶Q/H-⁷X-⁸A-⁹E-¹⁰X-¹¹I/V-¹²I-¹³X-¹⁴R | 15 |
| ¹S-²X-³A-⁴A-⁵X-⁶Q/H-⁷X-⁸X-⁹A-¹⁰X-¹¹I/V-¹²I-¹³X-¹⁴R | 16 |
| ¹S-²X-³X-⁴A-⁵E-⁶Q/H-⁷X-⁸X-⁹X-¹⁰S-¹¹I/V-¹²I-¹³X-¹⁴R | 17 |
| ¹S-²X-³X-⁴A-⁵A-⁶Q/H-⁷X-⁸X-⁹X-¹⁰A-¹¹I/V-¹²I-¹³X-¹⁴R | 18 |
| ¹S-²X-³X-⁴A-⁵X-⁶Q/H-⁷X-⁸X-⁹X-¹⁰X-¹¹I/V-¹²I-¹³H-¹⁴R | 19 |
| ¹S-²X-³X-⁴A-⁵X-⁶Q/H-⁷X-⁸X-⁹X-¹⁰X-¹¹I/V-¹²I-¹³A-¹⁴R | 20 |
| ¹S-²L-³G-⁴A-⁵X-⁶Q/H-⁷X-⁸X-⁹X-¹⁰X-¹¹I/V-¹²I-¹³X-¹⁴R | 21 |
| ¹S-²L-³G-⁴A-⁵E-⁶Q/H-⁷X-⁸X-⁹X-¹⁰X-¹¹I/V-¹²I-¹³X-¹⁴R | 22 |
| ¹S-²L-³G-⁴A-⁵A-⁶Q/H-⁷X-⁸X-⁹X-¹⁰X-¹¹I/V-¹²I-¹³X-¹⁴R | 23 |
| ¹S-²L-³G-⁴A-⁵X-⁶Q/H-⁷R-⁸X-⁹X-¹⁰X-¹¹I/V-¹²I-¹³X-¹⁴R | 24 |
| ¹S-²L-³G-⁴A-⁵X-⁶Q/H-⁷A-⁸X-⁹X-¹⁰X-¹¹I/V-¹²I-¹³X-¹⁴R | 25 |
| ¹S-²L-³G-⁴A-⁵X-⁶Q/H-⁷X-⁸T-⁹X-¹⁰X-¹¹I/V-¹²I-¹³X-¹⁴R | 26 |
| ¹S-²L-³G-⁴A-⁵X-⁶Q/H-⁷X-⁸A-⁹E-¹⁰X-¹¹I/V-¹²I-¹³X-¹⁴R | 27 |
| ¹S-²L-³G-⁴A-⁵X-⁶Q/H-⁷X-⁸X-⁹A-¹⁰X-¹¹I/V-¹²I-¹³X-¹⁴R | 28 |
| ¹S-²L-³G-⁴A-⁵X-⁶Q/H-⁷X-⁸X-⁹X-¹⁰S-¹¹I/V-¹²I-¹³X-¹⁴R | 29 |
| ¹S-²L-³G-⁴A-⁵X-⁶Q/H-⁷X-⁸X-⁹X-¹⁰A-¹¹I/V-¹²I-¹³X-¹⁴R | 30 |
| ¹S-²L-³G-⁴A-⁵X-⁶Q/H-⁷X-⁸X-⁹X-¹⁰S-¹¹I/V-¹²I-¹³H-¹⁴R | 31 |
| ¹S-²L-³G-⁴A-⁵X-⁶Q/H-⁷X-⁸X-⁹X-¹⁰A-¹¹I/V-¹²I-¹³A-¹⁴R | 32 |
| ¹S-²L-³G-⁴A-⁵E-⁶Q/H-⁷X-⁸X-⁹X-¹⁰X-¹¹I/V-¹²I-¹³X-¹⁴R | 33 |
| ¹S-²L-³G-⁴A-⁵E-⁶Q/H-⁷R-⁸X-⁹X-¹⁰X-¹¹I/V-¹²I-¹³X-¹⁴R | 34 |
| ¹S-²L-³G-⁴A-⁵E-⁶Q/H-⁷A-⁸X-⁹X-¹⁰X-¹¹I/V-¹²I-¹³X-¹⁴R | 35 |
| ¹S-²L-³G-⁴A-⁵E-⁶Q/H-⁷X-⁸T-⁹X-¹⁰X-¹¹I/V-¹²I-¹³X-¹⁴R | 36 |
| ¹S-²L-³G-⁴A-⁵E-⁶Q/H-⁷X-⁸A-⁹E-¹⁰X-¹¹I/V-¹²I-¹³X-¹⁴R | 37 |
| ¹S-²L-³G-⁴A-⁵E-⁶Q/H-⁷X-⁸X-⁹A-¹⁰X-¹¹I/V-¹²I-¹³X-¹⁴R | 38 |
| ¹S-²L-³G-⁴A-⁵E-⁶Q/H-⁷X-⁸X-⁹X-¹⁰S-¹¹I/V-¹²I-¹³X-¹⁴R | 39 |
| ¹S-²L-³G-⁴A-⁵E-⁶Q/H-⁷X-⁸X-⁹X-¹⁰A-¹¹I/V-¹²I-¹³X-¹⁴R | 40 |
| ¹S-²L-³G-⁴A-⁵E-⁶Q/H-⁷X-⁸X-⁹X-¹⁰S-¹¹I/V-¹²I-¹³H-¹⁴R | 41 |
| ¹S-²L-³G-⁴A-⁵E-⁶Q/H-⁷X-⁸X-⁹X-¹⁰A-¹¹I/V-¹²I-¹³A-¹⁴R | 42 |
| ¹S-²L-³G-⁴A-⁵E-⁶Q/H-⁷R-⁸X-⁹X-¹⁰X-¹¹I/V-¹²I-¹³X-¹⁴R | 43 |
| ¹S-²L-³G-⁴A-⁵E-⁶Q/H-⁷R-⁸T-⁹X-¹⁰X-¹¹I/V-¹²I-¹³X-¹⁴R | 44 |
| ¹S-²L-³G-⁴A-⁵E-⁶Q/H-⁷R-⁸A-⁹E-¹⁰X-¹¹I/V-¹²I-¹³X-¹⁴R | 45 |
| ¹S-²L-³G-⁴A-⁵E-⁶Q/H-⁷R-⁸X-⁹A-¹⁰X-¹¹I/V-¹²I-¹³X-¹⁴R | 46 |
| ¹S-²L-³G-⁴A-⁵E-⁶Q/H-⁷R-⁸X-⁹X-¹⁰S-¹¹I/V-¹²I-¹³X-¹⁴R | 47 |
| ¹S-²L-³G-⁴A-⁵E-⁶Q/H-⁷R-⁸X-⁹X-¹⁰A-¹¹I/V-¹²I-¹³X-¹⁴R | 48 |
| ¹S-²L-³G-⁴A-⁵E-⁶Q/H-⁷R-⁸X-⁹X-¹⁰S-¹¹I/V-¹²I-¹³H-¹⁴R | 49 |
| ¹S-²L-³G-⁴A-⁵E-⁶Q/H-⁷R-⁸X-⁹X-¹⁰A-¹¹I/V-¹²I-¹³A-¹⁴R | 50 |
| ¹S-²L-³G-⁴A-⁵E-⁶Q/H-⁷R-⁸T-⁹X-¹⁰X-¹¹I/V-¹²I-¹³X-¹⁴R | 51 |
| ¹S-²L-³G-⁴A-⁵E-⁶Q/H-⁷R-⁸T-⁹E-¹⁰X-¹¹I/V-¹²I-¹³X-¹⁴R | 52 |
| ¹S-²L-³G-⁴A-⁵E-⁶Q/H-⁷R-⁸T-⁹A-¹⁰X-¹¹I/V-¹²I-¹³X-¹⁴R | 53 |
| ¹S-²L-³G-⁴A-⁵E-⁶Q/H-⁷R-⁸T-⁹X-¹⁰S-¹¹I/V-¹²I-¹³X-¹⁴R | 54 |
| ¹S-²L-³G-⁴A-⁵E-⁶Q/H-⁷R-⁸T-⁹X-¹⁰A-¹¹I/V-¹²I-¹³X-¹⁴R | 55 |
| ¹S-²L-³G-⁴A-⁵E-⁶Q/H-⁷R-⁸T-⁹X-¹⁰S-¹¹I/V-¹²I-¹³H-¹⁴R | 56 |
| ¹S-²L-³G-⁴A-⁵E-⁶Q/H-⁷R-⁸T-⁹X-¹⁰A-¹¹I/V-¹²I-¹³A-¹⁴R | 57 |
| ¹S-²L-³G-⁴A-⁵E-⁶Q/H-⁷R-⁸T-⁹E-¹⁰S-¹¹I/V-¹²I-¹³X-¹⁴R | 58 |
| ¹S-²L-³G-⁴A-⁵E-⁶Q/H-⁷R-⁸T-⁹E-¹⁰A-¹¹I/V-¹²I-¹³X-¹⁴R | 59 |
| ¹S-²L-³G-⁴A-⁵E-⁶Q/H-⁷R-⁸T-⁹E-¹⁰X-¹¹I/V-¹²I-¹³H-¹⁴R | 60 |
| ¹S-²L-³G-⁴A-⁵E-⁶Q/H-⁷R-⁸T-⁹E-¹⁰X-¹¹I/V-¹²I-¹³A-¹⁴R | 61 |
| ¹S-²L-³G-⁴A-⁵E-⁶Q/H-⁷R-⁸T-⁹E-¹⁰S-¹¹I/V-¹²I-¹³X-¹⁴R | 62 |
| ¹S-²L-³G-⁴A-⁵E-⁶Q/H-⁷R-⁸T-⁹E-¹⁰S-¹¹I/V-¹²I-¹³A-¹⁴R | 63 |
| ¹S-²L-³G-⁴A-⁵E-⁶Q/H-⁷R-⁸T-⁹X-¹⁰S-¹¹I/V-¹²I-¹³H-¹⁴R | 64 |
| ¹S-²L-³G-⁴A-⁵E-⁶Q/H-⁷R-⁸T-⁹A-¹⁰S-¹¹I/V-¹²I-¹³H-¹⁴R | 65 |
| ¹S-²L-³G-⁴A-⁵E-⁶Q/H-⁷R-⁸X-⁹E-¹⁰S-¹¹I/V-¹²I-¹³H-¹⁴R | 66 |
| ¹S-²L-³G-⁴A-⁵E-⁶Q/H-⁷R-⁸A-⁹E-¹⁰S-¹¹I/V-¹²I-¹³H-¹⁴R | 67 |
| ¹S-²L-³G-⁴A-⁵E-⁶Q/H-⁷X-⁸T-⁹E-¹⁰S-¹¹I/V-¹²I-¹³H-¹⁴R | 68 |
| ¹S-²L-³G-⁴A-⁵E-⁶Q/H-⁷A-⁸T-⁹E-¹⁰S-¹¹I/V-¹²I-¹³H-¹⁴R | 69 |
| ¹S-²L-³G-⁴A-⁵X-⁶Q/H-⁷R-⁸T-⁹E-¹⁰S-¹¹I/V-¹²I-¹³H-¹⁴R | 70 |
| ¹S-²L-³G-⁴A-⁵A-⁶Q/H-⁷R-⁸T-⁹E-¹⁰S-¹¹I/V-¹²I-¹³H-¹⁴R | 71 |
| ¹S-²L-³X-⁴A-⁵E-⁶Q/H-⁷R-⁸T-⁹E-¹⁰S-¹¹I/V-¹²I-¹³H-¹⁴R | 72 |
| ¹S-²L-³A-⁴A-⁵E-⁶Q/H-⁷R-⁸T-⁹E-¹⁰S-¹¹I/V-¹²I-¹³H-¹⁴R | 73 |
| ¹S-²X-³G-⁴A-⁵E-⁶Q/H-⁷R-⁸T-⁹E-¹⁰S-¹¹I/V-¹²I-¹³H-¹⁴R | 74 |
| ¹S-²A-³G-⁴A-⁵E-⁶Q/H-⁷R-⁸T-⁹E-¹⁰S-¹¹I/V-¹²I-¹³H-¹⁴R | 75 |

### Effect of the 29-Mer Variants in Rat Model of Experimental OA

### Antinociceptive Effects of the 29-Mer PDSP Variants on MIA-Induced Hind Paw Weight-Bearing Shifts

In the animal study, the 29-mer PDSP variants were formulated into 1% HA to final a concentration of 0.2 mM and then applied by way of a single intraarticular injection each on day 1 and day 8 post-MIA injection. After 29-mer variant treatments for 14 days, the antinociceptive effects of the 29-mer variants (n=3 per group) on MIA-induced hind paw weight-bearing shifts were evaluated with an incapacitance tester.

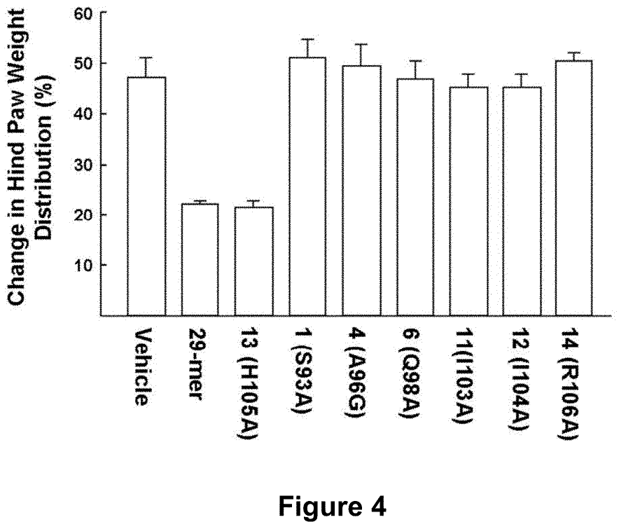

As shown in FIG. 4, the 29-mer/HA treatment significantly reduced the MIA-induced weight-bearing shifts, as compared to vehicle/HA treatment group (22.0±0.66% versus 47.1±3.7%; P<0.0004). H105A variant was also able to reduce MIA-induced weight shift (21.4±1.4%). Importantly, treatment with S93A, A96G, Q98A, 1103A, 1104A, and R106A variants had no effect on decreasing the MIA-induced hind paw weight-bearing shifts (values among 45-51%). The animal study results support that those critical residues play crucial role in sustaining the antinociceptive effects of 29-mer PDSP and that substitutions at the non-critical sites do not impact the activities of the PDSP.

### The Effects of the 29-Mer Variants on Sox9-Positive Chondrocyte Proliferation

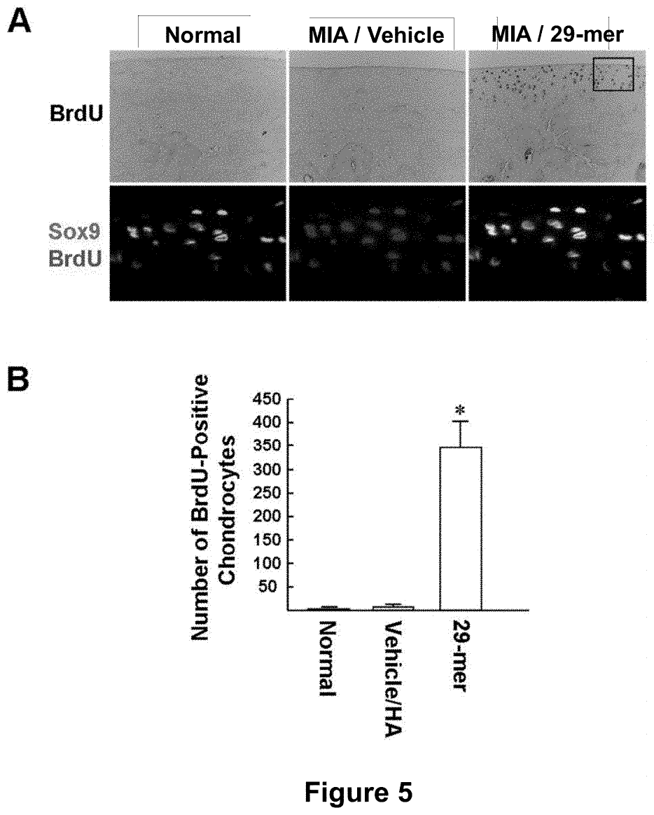

We started the BrdU treatment at day 1 after MIA injections for 7 days (set as day 0) to monitor cell proliferation immediately when treated with 0.2 mM 29-mer/HA. As shown in FIG. 5A (upper panels), almost all regenerative cartilage-like tissues contain BrdU-positive cells in the 29-mer/HA treatment group. However, there are few BrdU-positive cells on the cartilage surface of the vehicle/HA-treated knees, indicating that almost all chondrocytes are destined to necrotic cell death after MIA treatment.

Transcription factor Sox9 plays an essential role in stem/progenitor cell chondrogenesis by directing the expression of chondrocyte-specific genes.

Immunostaining of Sox9 found that the BrdU-positive cells were also Sox9 positive, indicating that the BrdU-positive cells were induced by the 29-mer potentially toward chondrocyte development (FIG. 5A; lower panels). 0.2 mM 29-mer treatment displayed a significant ability to induce expansion of BrdU-positive chondrocytes in regenerative cartilage, as compared to vehicle/HA treatment (FIG. 5B; 346±57 versus 7±3; P<0.001). Collectively, these results indicate that the 29-mer can induce a chondrogenic cell proliferation to heal cartilage.

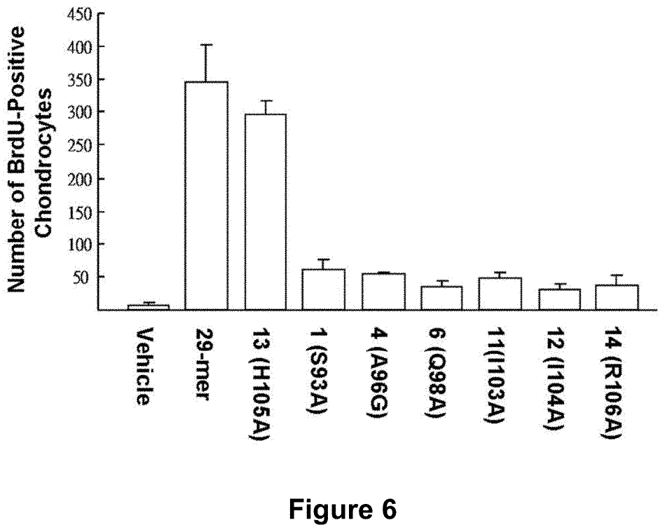

Next, we examined the ability of the 29-mer variants to induce cell proliferation on articular cartilage after the 29-mer treatment for 14 days. BrdU-immunostaining of knee joints revealed that numerous BrdU-positive cells were detectable in the cartilage regions of the 29-mer/HA- and H105A/HA-treated groups, whereas those from vehicle/HA treatment contained fewer BrdU-positive cells (FIG. 6; 346±57 and 297±22 versus 7±3). On the other hand, treatment with S93A, A96G, Q98A, 1103A, 1104A, and R106A did not increase BrdU-positive cells at cartilage (values among 30-62). This animal study confirmed that those critical residues play crucial roles in maintaining 29-mer PDSP biological activity.

Taken together, alanine scanning data indicate that the therapeutic effect of the 29-mer is influenced by selected amino acid substitution, as evidenced by rat model of osteoarthritis. Moreover, the 29-mer residues at positions S93, A96, Q98, 1103, 1104, and R106 are important for the 29-mer PDSP activity in OA treatment, whereas other residues can be substituted without significant impact on the activities.

Embodiments of the invention have been illustrated with a limited number of examples. One skilled in the art would appreciate that variations and modifications are possible without departing from the scope of the invention. Therefore, the scope of the invention should only be limited by the accompanied claims.

What is claimed is:

1. A pharmaceutical composition for use in treating and/or preventing osteoarthritis, comprising: a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

2. The pharmaceutical composition according to claim 1, wherein the PDSP comprises the sequence of S-X-X-AX-Q/H-X-X-X-X-I/V-I-X-R (SEQ ID NO:5).

3. The pharmaceutical composition according to claim 1, wherein the PDSP comprises the sequence of SLGAEQRTESIIHR (SEQ ID NO:2).

4. The pharmaceutical composition according to claim 1, wherein the PDSP comprises the sequence of SLGAEQRTESIIHRALYYDLISSPDIHGT (SEQ ID NO:1).

5. The pharmaceutical composition according to claim 1, wherein the PDSP comprises the sequence of the sequences of any one of SEQ ID NO: 6 to 75.

6. A pharmaceutical composition for promoting chondrogenesis, comprising: a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

7. The pharmaceutical composition according to claim 6, wherein the PDSP comprises the sequence of S-X-X-A-X-Q/H-X-X-X-X-I/V-I-X-R (SEQ ID NO:5).

8. The pharmaceutical composition according to claim 6, wherein the PDSP comprises the sequence of SLGAEQRTESIIHR (SEQ ID NO:2).

9. The pharmaceutical composition according to claim 6, wherein the PDSP comprises the sequence of SLGAEQRTESIIHRALYYDLISSPDIHGT (SEQ ID NO:1).

10. The pharmaceutical composition according to claim 6, wherein the PDSP comprises the sequence of the sequences of any one of SEQ ID NO: 6 to 75.


SEQUENCE LISTING (75 sequences)

SEQ ID NO: 1 - protein, 29 aa, Homo sapiens
SLGAEQRTESIIHRALYYDLISSPDIHGT

SEQ ID NO: 2 - protein, 14 aa, Homo sapiens
SLGAEQRTESIIHR

SEQ ID NO: 3 - protein, 29 aa, Mus musculus
SLGAEHRTESVIHRALYYDLITNPDIHST

SEQ ID NO: 4 - protein, 14 aa, Mus musculus
SLGAEHRTESVIHR

SEQ ID NO: 5 - protein, 14 aa, Artificial Sequence
  synthetic
  misc_feature (2)..(3): Xaa can be any naturally occurring amino acid
  misc_feature (5)..(11): Xaa can be any naturally occurring amino acid
  misc_feature (13)..(13): Xaa can be any naturally occurring amino acid
SXXAXXXXXXXIXR

SEQ ID NO: 6 - protein, 14 aa, Artificial Sequence
  synthetic
  misc_feature (3)..(3): Xaa can be any naturally occurring amino acid
  misc_feature (5)..(11): Xaa can be any naturally occurring amino acid
  misc_feature (13)..(13): Xaa can be any naturally occurring amino acid
SLXAXXXXXXXIXR

SEQ ID NO: 7 - protein, 14 aa, Artificial Sequence
  synthetic
  misc_feature (3)..(3): Xaa can be any naturally occurring amino acid
  misc_feature (5)..(11): Xaa can be any naturally occurring amino acid
  misc_feature (13)..(13): Xaa can be any naturally occurring amino acid
SAXAXXXXXXXIXR

SEQ ID NO: 8 - protein, 14 aa, Artificial Sequence
  synthetic
  misc_feature (2)..(2): Xaa can be any naturally occurring amino acid
  misc_feature (5)..(11): Xaa can be any naturally occurring amino acid
  misc_feature (13)..(13): Xaa can be any naturally occurring amino acid
SXGAXXXXXXXIXR

SEQ ID NO: 9 - protein, 14 aa, Artificial Sequence
  synthetic
  misc_feature (2)..(2): Xaa can be any naturally occurring amino acid
  misc_feature (5)..(11): Xaa can be any naturally occurring amino acid
  misc_feature (13)..(13): Xaa can be any naturally occurring amino acid
SXAAXXXXXXXIXR

SEQ ID NO: 10 - protein, 14 aa, Artificial Sequence
  synthetic
  misc_feature (2)..(3): Xaa can be any naturally occurring amino acid
  misc_feature (6)..(11): Xaa can be any naturally occurring amino acid
  misc_feature (13)..(13): Xaa can be any naturally occurring amino acid
SXXAEXXXXXXIXR

SEQ ID NO: 11 - protein, 14 aa, Artificial Sequence
  synthetic
  misc_feature (2)..(3): Xaa can be any naturally occurring amino acid
  misc_feature (6)..(11): Xaa can be any naturally occurring amino acid
  misc_feature (13)..(13): Xaa can be any naturally occurring amino acid
SXXAAXXXXXXIXR

SEQ ID NO: 12 - protein, 14 aa, Artificial Sequence
  synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SXXAXXRXXXXIXR

SEQ ID NO: 13 - protein, 14 aa, Artificial Sequence
  synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SXXAXXAXXXXIXR

SEQ ID NO: 14 - protein, 14 aa, Artificial Sequence
  synthetic
  misc_feature (2)..(3): Xaa can be any naturally occurring amino acid
  misc_feature (5)..(7): Xaa can be any naturally occurring amino acid
  misc_feature (9)..(11): Xaa can be any naturally occurring amino acid
  misc_feature (13)..(13): Xaa can be any naturally occurring amino acid
SXXAXXXTXXXIXR

SEQ ID NO: 15 - protein, 14 aa, Artificial Sequence
  synthetic
  misc_feature (2)..(3): Xaa can be any naturally occurring amino acid
  misc_feature (5)..(7): Xaa can be any naturally occurring amino acid
  misc_feature (10)..(11): Xaa can be any naturally occurring amino acid
  misc_feature (13)..(13): Xaa can be any naturally occurring amino acid
SXXAXXXAEXXIXR

SEQ ID NO: 16 - protein, 14 aa, Artificial Sequence
  synthetic
  misc_feature (2)..(3): Xaa can be any naturally occurring amino acid
  misc_feature (5)..(8): Xaa can be any naturally occurring amino acid
  misc_feature (10)..(11): Xaa can be any naturally occurring amino acid
  misc_feature (13)..(13): Xaa can be any naturally occurring amino acid
SXXAXXXXAXXIXR

SEQ ID NO: 17 - protein, 14 aa, Artificial Sequence
  synthetic
  misc_feature (2)..(3): Xaa can be any naturally occurring amino acid
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SXXAXXXXXSXIXR

SEQ ID NO: 18 - protein, 14 aa, Artificial Sequence
  synthetic
  misc_feature (2)..(3): Xaa can be any naturally occurring amino acid
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SXXAXXXXXAXIXR

SEQ ID NO: 19 - protein, 14 aa, Artificial Sequence
  synthetic
  misc_feature (2)..(3): Xaa can be any naturally occurring amino acid
  misc_feature (5)..(11): Xaa can be any naturally occurring amino acid
SXXAXXXXXXXIHR

SEQ ID NO: 20 - protein, 14 aa, Artificial Sequence
  synthetic
  misc_feature (2)..(3): Xaa can be any naturally occurring amino acid
  misc_feature (5)..(11): Xaa can be any naturally occurring amino acid
SXXAXXXXXXXIAR

SEQ ID NO: 21 - protein, 14 aa, Artificial Sequence
  synthetic
  misc_feature (5)..(11): Xaa can be any naturally occurring amino acid
  misc_feature (13)..(13): Xaa can be any naturally occurring amino acid
SLGAXXXXXXXIXR

SEQ ID NO: 22 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAEXXXXXXIXR

SEQ ID NO: 23 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAAXXXXXXIXR

SEQ ID NO: 24 - protein, 14 aa, Artificial Sequence
  synthetic
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SLGAXXRXXXXIXR

SEQ ID NO: 25 - protein, 14 aa, Artificial Sequence
  synthetic
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SLGAXXAXXXXIXR

SEQ ID NO: 26 - protein, 14 aa, Artificial Sequence
  synthetic
  misc_feature (5)..(7): Xaa can be any naturally occurring amino acid
  misc_feature (9)..(11): Xaa can be any naturally occurring amino acid
  misc_feature (13)..(13): Xaa can be any naturally occurring amino acid
SLGAXXXTXXXIXR

SEQ ID NO: 27 - protein, 14 aa, Artificial Sequence
  synthetic
  misc_feature (5)..(7): Xaa can be any naturally occurring amino acid
  misc_feature (10)..(11): Xaa can be any naturally occurring amino acid
  misc_feature (13)..(13): Xaa can be any naturally occurring amino acid
SLGAXXXAEXXIXR

SEQ ID NO: 28 - protein, 14 aa, Artificial Sequence
  synthetic
  misc_feature (5)..(8): Xaa can be any naturally occurring amino acid
  misc_feature (10)..(11): Xaa can be any naturally occurring amino acid
  misc_feature (13)..(13): Xaa can be any naturally occurring amino acid
SLGAXXXXAXXIXR

SEQ ID NO: 29 - protein, 14 aa, Artificial Sequence
  synthetic
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SLGAXXXXXSXIXR

SEQ ID NO: 30 - protein, 14 aa, Artificial Sequence
  synthetic
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SLGAXXXXXAXIXR

SEQ ID NO: 31 - protein, 14 aa, Artificial Sequence
  synthetic
  misc_feature (5)..(9): Xaa can be any naturally occurring amino acid
  misc_feature (11)..(11): Xaa can be any naturally occurring amino acid
SLGAXXXXXSXIHR

SEQ ID NO: 32 - protein, 14 aa, Artificial Sequence
  synthetic
  misc_feature (5)..(9): Xaa can be any naturally occurring amino acid
  misc_feature (11)..(11): Xaa can be any naturally occurring amino acid
SLGAXXXXXAXIAR

SEQ ID NO: 33 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAEXXXXXXIXR

SEQ ID NO: 34 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXRXXXXIXR

SEQ ID NO: 35 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXAXXXXIXR

SEQ ID NO: 36 - protein, 14 aa, Artificial Sequence
  synthetic
  misc_feature (6)..(7): Xaa can be any naturally occurring amino acid
  misc_feature (9)..(11): Xaa can be any naturally occurring amino acid
  misc_feature (13)..(13): Xaa can be any naturally occurring amino acid
SLGAEXXTXXXIXR

SEQ ID NO: 37 - protein, 14 aa, Artificial Sequence
  synthetic
  misc_feature (6)..(7): Xaa can be any naturally occurring amino acid
  misc_feature (10)..(11): Xaa can be any naturally occurring amino acid
  misc_feature (13)..(13): Xaa can be any naturally occurring amino acid
SLGAEXXAEXXIXR

SEQ ID NO: 38 - protein, 14 aa, Artificial Sequence
  synthetic
  misc_feature (6)..(8): Xaa can be any naturally occurring amino acid
  misc_feature (10)..(11): Xaa can be any naturally occurring amino acid
  misc_feature (13)..(13): Xaa can be any naturally occurring amino acid
SLGAEXXXAXXIXR

SEQ ID NO: 39 - protein, 14 aa, Artificial Sequence
  synthetic
  misc_feature (6)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXXXXSXIXR

SEQ ID NO: 40 - protein, 14 aa, Artificial Sequence
  synthetic
  misc_feature (6)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXXXXAXIXR

SEQ ID NO: 41 - protein, 14 aa, Artificial Sequence
  synthetic
  misc_feature (6)..(9): Xaa can be any naturally occurring amino acid
  misc_feature (11)..(11): Xaa can be any naturally occurring amino acid
SLGAEXXXXSXIHR

SEQ ID NO: 42 - protein, 14 aa, Artificial Sequence
  synthetic
  misc_feature (6)..(9): Xaa can be any naturally occurring amino acid
  misc_feature (11)..(11): Xaa can be any naturally occurring amino acid
SLGAEXXXXAXIAR

SEQ ID NO: 43 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXRXXXXIXR

SEQ ID NO: 44 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRTXXXIXR

SEQ ID NO: 45 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRAXXXIXR

SEQ ID NO: 46 - protein, 14 aa, Artificial Sequence
  synthetic
  misc_feature (6)..(6): Xaa can be any naturally occurring amino acid
  misc_feature (8)..(8): Xaa can be any naturally occurring amino acid
  misc_feature (10)..(11): Xaa can be any naturally occurring amino acid
  misc_feature (13)..(13): Xaa can be any naturally occurring amino acid
SLGAEXRXAXXIXR

SEQ ID NO: 47 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRXXSXIXR

SEQ ID NO: 48 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRXXAXIXR

SEQ ID NO: 49 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
SLGAEXRXXSXIHR

SEQ ID NO: 50 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
SLGAEXRXXAXIAR

SEQ ID NO: 51 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRTXXXIXR

SEQ ID NO: 52 - protein, 14 aa, Artificial Sequence
  synthetic
  misc_feature (6)..(6): Xaa can be any naturally occurring amino acid
  misc_feature (10)..(11): Xaa can be any naturally occurring amino acid
  misc_feature (13)..(13): Xaa can be any naturally occurring amino acid
SLGAEXRTEXXIXR

SEQ ID NO: 53 - protein, 14 aa, Artificial Sequence
  synthetic
  misc_feature (6)..(6): Xaa can be any naturally occurring amino acid
  misc_feature (10)..(11): Xaa can be any naturally occurring amino acid
  misc_feature (13)..(13): Xaa can be any naturally occurring amino acid
SLGAEXRTAXXIXR

SEQ ID NO: 54 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRTXSXIXR

SEQ ID NO: 55 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRTXAXIXR

SEQ ID NO: 56 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXSXIHR

SEQ ID NO: 57 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXAXIAR

SEQ ID NO: 58 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTESXIXR

SEQ ID NO: 59 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTEAXIXR

SEQ ID NO: 60 - protein, 14 aa, Artificial Sequence
  synthetic
  misc_feature (6)..(6): Xaa can be any naturally occurring amino acid
  misc_feature (10)..(11): Xaa can be any naturally occurring amino acid
SLGAEXRTEXXIHR

SEQ ID NO: 61 - protein, 14 aa, Artificial Sequence
  synthetic
  misc_feature (6)..(6): Xaa can be any naturally occurring amino acid
  misc_feature (10)..(11): Xaa can be any naturally occurring amino acid
SLGAEXRTEXXIAR

SEQ ID NO: 62 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTESXIXR

SEQ ID NO: 63 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRTESXIAR

SEQ ID NO: 64 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXSXIHR

SEQ ID NO: 65 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRTASXIHR

SEQ ID NO: 66 - protein, 14 aa, Artificial Sequence
  synthetic
  misc_feature (6)..(6): Xaa can be any naturally occurring amino acid
  misc_feature (8)..(8): Xaa can be any naturally occurring amino acid
  misc_feature (11)..(11): Xaa can be any naturally occurring amino acid
SLGAEXRXESXIHR

SEQ ID NO: 67 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRAESXIHR

SEQ ID NO: 68 - protein, 14 aa, Artificial Sequence
  synthetic
  misc_feature (6)..(7): Xaa can be any naturally occurring amino acid
  misc_feature (11)..(11): Xaa can be any naturally occurring amino acid
SLGAEXXTESXIHR

SEQ ID NO: 69 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXATESXIHR

SEQ ID NO: 70 - protein, 14 aa, Artificial Sequence
  synthetic
  misc_feature (5)..(6): Xaa can be any naturally occurring amino acid
  misc_feature (11)..(11): Xaa can be any naturally occurring amino acid
SLGAXXRTESXIHR

SEQ ID NO: 71 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAAXRTESXIHR

SEQ ID NO: 72 - protein, 14 aa, Artificial Sequence
  synthetic
  misc_feature (3)..(3): Xaa can be any naturally occurring amino acid
  misc_feature (6)..(6): Xaa can be any naturally occurring amino acid
  misc_feature (11)..(11): Xaa can be any naturally occurring amino acid
SLXAEXRTESXIHR

SEQ ID NO: 73 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLAAEXRTESXIHR

SEQ ID NO: 74 - protein, 14 aa, Artificial Sequence
  synthetic
  misc_feature (2)..(2): Xaa can be any naturally occurring amino acid
  misc_feature (6)..(6): Xaa can be any naturally occurring amino acid
  misc_feature (11)..(11): Xaa can be any naturally occurring amino acid
SXGAEXRTESXIHR

SEQ ID NO: 75 - protein, 14 aa, Artificial Sequence
  synthetic
  misc_feature (6)..(6): Xaa can be any naturally occurring amino acid
  misc_feature (11)..(11): Xaa can be any naturally occurring amino acid
SAGAEXRTESXIHR